(19) United States
(12) Patent Application Publication
(10) Pub. No.: US 2013/0237774 A1
(43) Pub. Date: Sep. 12, 2013

(54) Device and Method for Continuous Chemical Sensing

(71) Applicants: Jerome J. Schentag, Eggertsville, NY (US); Frank V. Bright, Williamsville, NY (US); David T. D'Andrea, Getzville, NY (US)

(72) Inventors: Jerome J. Schentag, Eggertsville, NY (US); Frank V. Bright, Williamsville, NY (US); David T. D'Andrea, Getzville, NY (US)

(73) Assignee: THERASYN SENSORS, INC., Amherst, NY (US)

(21) Appl. No.: 13/808,453

(22) PCT Filed: Jul. 7, 2011
(86) PCT No.: PCT/US2011/043237

§ 371 Date: May 22, 2013

Related U.S. Application Data
(60) Provisional application No. 61/362,149, filed on Jul. 7, 2010.
(60) Provisional application No. 61/364,820, filed on Jul. 16, 2010.

(51) Int. Cl. A61B 5/07; A61B 1/04; A61B 1/00; A61B 5/00; A61B 7/00; A61B 1/06; A61B 5/1459; A61B 5/145; A61B 5/03

(52) U.S. Cl. CPC A61B 5/073; A61B 5/14507; A61B 1/041; A61B 1/00011; A61B 5/14539; A61B 5/036; A61B 7/008; A61B 1/0661; A61B 5/1459; A61B 5/0004
USPC 600/301; 600/302

(57) ABSTRACT

The present invention may be embodied as an ingestible device capable of sensing one or more chemical parameters. In use, the device can continuously determine the chemical concentrations within an alimentary canal tract. An embodiment of the device comprises a housing resistant to degradation by alimentary canal fluid, a light source, and image capture device. An analyte sensor is configured to obtain at least one measurement of a concentration of analyte in the fluid. The analyte sensor comprises a sensor substance in a sol-gel material so the sensor substance reversibly interacts with an analyte of interest. In addition, the analyte sensor is configured to generate a trigger signal for controlling the operation of subsystems in the device.

### CROSS-REFERENCE TO RELATED APPLICATION

This application claims the benefit of priority to U.S. provisional patent application Ser. No. 61/362,149 filed Jul. 7, 2010, and to U.S. provisional patent application Ser. No. 61/364,820 filed Jul. 16, 2010, the disclosures of which are incorporated herein by reference.

### FIELD OF THE INVENTION

The present invention relates to a device for monitoring the alimentary canal, including using signals from a chemical sensor to trigger and control the operation of the device and its subsystems, and more specifically, to a measurement device capable of being disposed in a fluid.

### BACKGROUND OF THE INVENTION

In recent years, a non-digestible capsule, containing sensors and a radio transmitter, has been seen by the medical profession as a possible way to monitor various body environments. The ideal ingestible capsule is seen as one that is small enough to be easily ingested, biologically inert, disposable, and inexpensive. The transmission signal would have to be sufficiently strong to be received by a remote receiver, preferably located apart from the patient's body so that the patient would have freedom of movement, or, be small enough to be carried by the patient.

Previous ingestible capsules have not used continuous sensing because chemical means of capture have not been applied to these capsules. Intestinal fluids degrade antibody capture sensing methods, and these are not capable of measuring continuously. Previous ingestible capsules did not envision optical detection systems for use in tracking distance travelled in the alimentary canal and for the continuous sensing of chemicals, including without limitation proteins, comprising the fluids in the alimentary canal.

### BRIEF SUMMARY OF THE INVENTION

A device according to an embodiment of the present invention is configured for measuring the concentration of an analyte in an alimentary fluid. The device comprises a housing that is resistant to degradation by alimentary fluid, a first light source configured to illuminate a region of the environment external to the housing, and a first image capture device disposed within the housing. The first image device is positioned to capture an image (or multiple images) of at least a portion of a first field-of-view.

The device has an analyte sensor capable of measuring the concentration of an analyte within the alimentary fluid, configured to obtain a plurality of measurements of the concentration of an analyte in the alimentary fluid. The analyte sensor comprises a sensor substance in a sol-gel material. The sensor substance is configured to reversibly interact with an analyte of interest. When the sensor substance is in contact with the analyte of interest and electromagnetic excitation energy is received by the sensor substance, the sensor substance will emit electromagnetic energy. The analyte sensor of a device of the present invention is configured to generate a signal for controlling the device.

Multiple sensor substances may be used. For example, an array may be formed from a plurality of sensor substances, each configured to respond to a different analyte of interest. In this way, multiple chemical parameters (i.e., concentrations of multiple analytes) may be measured simultaneously.

The invention may also be embodied as a method of repeatedly determining an analyte concentration in a fluid. A device configured to be disposed in the fluid is provided. The device comprises a sensor substance in a sol-gel material, an electromagnetic energy source capable of emitting light, and a detector configured to detect electromagnetic energy emitted by the sensor substance. The device is moved through at least a portion of the fluid. The sensor substance is exposed to the fluid. The detector is used to make at least one measurement of a property of the electromagnetic energy emitted by the sensor substance. A processor is used to determine at least one analyte concentration value of the fluid based on the plurality of measured properties. Measurements of emitted electromagnetic energy may be made continuously.

The invention may also be embodied as a method of determining a location of an ingestible device within an alimentary canal. The device would comprise an analyte sensor as described herein. The analyte sensor is used to determine a concentration of an analyte in a fluid proximate to the device. The location of the ingestible device within the alimentary canal is calculated based on the determined analyte concentration.

The method can further comprise using an accelerometer equipped device to determine a first acceleration of the device. The accelerometer is used to determine a second acceleration of the device. The second acceleration determination is made after the first acceleration and corresponds to a determined analyte concentration. A relative position of the device is determined based on the first and second acceleration. The relative position of the device is used in the calculation of the device location in the alimentary canal.

The present invention can also be embodied as a method of locating a region of interest within an alimentary canal using an ingestible device. In this method, the ingestible device has an analyte sensor and an accelerometer. The analyte sensor is used to determine a concentration of an analyte in a fluid proximate to the device. The analyte sensor sends a signal when the analyte sensor senses a concentration of the analyte greater than a pre-determined threshold value, and the pre-determined threshold value corresponding to a region of interest. The accelerometer and the analyte sensor are used to calculate the location of the device within the alimentary canal to determine the location of the region of interest.

### DESCRIPTION OF THE DRAWINGS

For a fuller understanding of the nature and objects of the invention, reference should be made to the following detailed description taken in conjunction with the accompanying drawings, in which:

FIG. 1 is a simplified block diagram of a circuit according to an embodiment of the present invention;

FIG. 2a is a top side view of the rigid flex assembly shown unfolded of another embodiment of the present invention;

FIG. 2b is a bottom side view of the rigid flex assembly shown unfolded in the same embodiment of the present invention as FIG. 2a;

FIG. 3 is a folded rigid flex assembly of an embodiment of the present invention before the case is installed or assembly is potted;

FIG. 4 illustrates a possible outer geometry for an embodiment of the device after encapsulation;

FIG. 5 is one possible configuration for the sensing cell of the present invention;

FIG. 6 illustrates an embodiment of a charge/activation stand for the device of the present invention;

FIG. 7 is a block diagram illustrating an embodiment of a method according to the present invention;

FIG. 8 is a block diagram illustrating another embodiment of a method according to the present invention; and

FIG. 9 is a block diagram illustrating another embodiment of a method according to the present invention.

### DETAILED DESCRIPTION OF THE INVENTION

A device 35 according to an embodiment of the present invention is configured for measuring the concentration of an analyte in an alimentary fluid. The device 35 comprises a housing 102 that is resistant to degradation by alimentary fluid. As such, the housing 102 allows the device 35 to pass through the alimentary canal of a subject without degradation such that measurements can be made and/or data may be captured at any location throughout the alimentary canal. It should be noted that the terms “alimentary tract fluid,” “gastrointestinal tract fluid” are used interchangeably throughout this disclosure, and should be given the broadest interpretation as at least any and all fluids, or components of these fluids, found in the alimentary canal of a living being. Similarly, the terms “alimentary canal,” “digestive tract,” and “gastrointestinal tract” are used interchangeably throughout this disclosure, and should be also be interpreted broadly. It should be noted that the composition of alimentary tract fluid and conditions of the alimentary tract change with location. For example, the physical and chemical nature of the alimentary tract fluid is dependent on the location of the capsule at the time of its travel. These changes can be measured through wide swings in pH (from 1.0 to 7.5), and the presence of location indicative digestive enzymes and proteases. For example, the stomach may have a pH of 1.0 and the duodenum with a pH of 5.5. Although the distance between the stomach and duodenum is mere inches, the physical pH value differs greatly.

The device 35 may be configured to be swallowed by a subject. For example, a device 35 according to an embodiment of the present invention may be capsule shaped.

The device 35 has a first light source 4. The first light source 4 is configured to illuminate a region of the environment external to the housing—a first “field-of-view.” The first light source 4 may be a light-emitting diode (“LED”). The first light source 4 may be located within the housing 102 such that the housing 102 protects the first light source 4 from degradation by alimentary fluid. In such embodiments, the housing 102, or at least a portion of the housing 102, may be transmissive so that the light from the first light source 4 can pass through the housing 102. The first light source 102 may be configured to illuminate a field-of-view at the leading end of the device 35 (when the device 35 is configured to have ends—e.g., a capsule shape). Alternatively, the first light source 102 may be configured to illuminate a field-of-view at a trailing end of the device 35, or a field-a-view at a side of the device 35. The field-of-view may be wide or narrow as suited to the purpose of the device 35. The illumination may be of any brightness and color temperature as suited to the purpose of the device 35.

The device 35 includes a first image capture device 2 disposed within the housing 102. The first image device 2 may be, for example, a still camera, a video camera, or a camera capable of both still image capture and video capture. The first image device 2 may be, for example, capable of capturing three-dimensional image information. For example, the first image device 2 may comprise multiple image sensors spaced apart from each other at a fixed distance. In this way, each image sensor will capture a view of the scene from a different perspective, and the perspective images can be merged to provide three-dimensional image data. The first image device 2 may be, for example, an infrared camera and/or a visible light camera. The first image device 2 is positioned to capture an image (or multiple images) of at least a portion of the first field-of-view. The housing 102 is transmissive in order to allow light to pass through to the first image device 2. For example, at least a portion of the housing 102 may be clear (i.e., transparent). In an embodiment, a portion of the housing 102 may be shaped in order to act as a lens for the first image device 2. The lens may be configured to show a magnified view, a wide-angle view, or otherwise. In certain cases, the lens may distort the optical view of the image capture device, for example, the lens may be a so-called “fish-eye” lens capable of a wide field-of-view, but distorting the image. A portion of the housing 102 may be configured to act as a filter in order to filter certain wavelengths of light from reaching the first image device 2.

The device 35 has an analyte sensor 11 capable of measuring the concentration of an analyte within the alimentary fluid. For example, the analyte sensor may be capable of measuring relatively small molecules (e.g., glucose) or relatively large molecules (e.g., proteins—hemoglobin). The capabilities, function, and structure of analyte sensors 11 is described in further detail infra. The analyte sensor 11 of a device 35 of the present invention is configured to obtain a plurality of measurements of the concentration of an analyte in the alimentary fluid. The analyte sensor comprises a sensor substance 30 in a sol-gel material. Sol-gel materials include materials derived from a sol-gel process. The sensor substance 30 is configured to reversibly interact with an analyte of interest. The sensor substance 30 may be able to sample an analyte at a rate of approximately one second or less, depending on the analyte and configuration of the analyte sensor 11. The sampling rate may be higher for larger molecules, or lower for smaller molecules. In addition, the analyte sensor itself may be disposed within the housing or external to the housing.

When the sensor substance 30 is in contact with the analyte of interest and electromagnetic excitation energy is received by the sensor substance 30, the sensor substance will emit electromagnetic energy. For example, the sensor substance may be exposed to electromagnetic excitation energy in the form of light energy. Such a sensor substance is also configured to react to a specific analyte of interest (e.g., glucose). When such a sensor substance is exposed to the analyte of interest and the excitation energy, the sensor substance will emit energy, for example the sensor substance may fluoresce. Other forms of electromagnetic excitation energy (e.g., infrared, ultraviolet, etc.) can by used. The sensor substance 30 can be configured to emit energy in different ways and in different forms. For example, the sensor substance can be configured to emit modulated light energy, or energy of specific wavelengths.

Multiple sensor substances may be used. For example, an array may be formed from a plurality of sensor substances, each configured to respond to a different analyte of interest. In this way, multiple chemical parameters (i.e., concentrations of multiple analytes) may be measured simultaneously.

The sensor substance 30 of a device 35 of the present invention is configured to be in contact with the alimentary fluid. In an embodiment, the sensor substance may be located within the external bounds of the device 35 generally defined by the housing 102. In such an embodiment, portions of the housing 102 may include one or more apertures through which alimentary fluid may move to contact the sensor substance 30. A cover material, such as, but not limited to, a membrane or a mesh, may cover the apertures and allow the alimentary fluid to pass through. In another embodiment, the sensor substance 30 is located on the housing such that the sensor substance 30 is exposed to the alimentary fluid without the fluid passing into the device 35.

The analyte sensor 11 of a device 35 of the present invention is configured to generate a signal for controlling the device 35. In an embodiment, the analyte sensor 11 is configured to generate a signal to the first image capture device 2, such that the first image capture device 2 will capture an image. In one example, the analyte sensor 11 may be configured to detect hemoglobin and trigger the first image capture device 2 to capture a plurality of images. Such an embodiment is useful for detecting and photographing portions of the digestive tract which may be bleeding. Other sensor configurations are possible and within the scope of the present invention.

The analyte sensor 11 of a device 35 of the present invention may also be configured to continuously measure a concentration of analyte in the alimentary fluid. In an embodiment, the analyte sensor 11 is configured to be reversible. The term “reversible” or “reversibility” as used herein refers to the ability of the analyte sensor to detect the presence of an analyte within a sample in a continuous manner as the sample concentration within the sample increases and decreases and to do so in an unbiased manner. The presence of the analyte is identified by detecting a signal that is indicative of the analyte concentration. The absence of the analyte can be identified by a lack of a detectable signal or a signal that is not significantly different than the background signal. Upon re-exposure to the analyte the signal can again be recorded. Any change over time in the concentration of the analyte in the immediate environment of the sensor results in a signal from the sensor that is readily correlated to the analyte concentration in the sample at the point in time of the signal measurement. The signal is also an accurate and precise measure of the analyte concentration at that specific point in time. The reversible nature of the interaction between the sensor and the analyte allows detection of an analyte in a continuous manner and no change in temperature or pressure or other means (e.g., pH swing, chaotrope, denaturant) is required to disengage/dissociate the analyte from the sensor. We have successfully used the present method for reversibly and continuously detecting analytes over a period of several months. For example, the signal from the chemical sensor was continuously detected over a period of at least one year with minimal drift (relative standard deviation ≦5%). The reversible nature of the analyte sensor 11 can be performed through physical or chemical means. For example, an optical fiber brush may be employed to clear the active surface of the analyte sensor between measurements. In another example, the analyte sensor 11 can be configured to detect the analyte as it flows through the sensor without association.

The device 35 may also comprise a detector 8 configured to detect electromagnetic energy emitted by the sensor substance 30. In an embodiment, the detector 8 is a CMOS detector that monitors the emitted energy. In another embodiment, the detector 8 is an array of CMOS detectors. Configuring CMOS detectors to react to certain wavelengths of light is well-known in the art. The device 35 may also comprise a controller 5 in electronic communication with the detector 8 for measuring a concentration of analyte based on the detected electromagnetic energy. The controller may compare the detected electromagnetic energy with a known value, or may calculate analyte concentration based on an algorithm specific to the known electromagnetic response.

The sensor substance 30 requires electromagnetic excitation energy in order to respond when in the presence of an analyte. In an embodiment, electromagnetic excitation energy is provided to the sensor substance by the first light source 4. If additional light sources are employed, these light sources may also provide the required electromagnetic excitation energy. The sensor substance 30 may receive the energy through ambient radiation, or the energy may be directed as, for example, a light channel through a fiber-optic cable. In another embodiment, the analyte sensor 11 further comprises an electromagnetic excitation energy source 9 configured to provide electromagnetic excitation energy to the sensor substance. For example, the source may be a driving LED configured to emit a specific wavelength, or wavelength range of light. The driving LED may also produce a modulated signal to aid in the computation of analyte concentration

The device 35 may further comprise a transmitter and/or receiver. The transmitter and/or receiver are at least in electronic communication with the first image capture device 2. In an embodiment, a transceiver is used configured to transmit collected data. The transceiver may also accept commands for control of the capsule's systems. It should be noted that the terms “transceiver” and “transmitter and/or receiver” are used interchangeably throughout this disclosure, and should be given the broadest interpretation as at least any device configured to transmit and/or receive information. In another embodiment, an antenna 13 is provided in communication with the transmitter and/or receiver. For example, the antenna 13 comprises a fractional wavelength pattern. The pattern may be etched on a thin sheet of Kapton™. In another embodiment, the transceiver function could be implemented using an 802.11a compatible device or a BlueTooth™ module.

The device 35 may further comprise a parametric sensor for measuring a physical parameter of the environment external to the housing. For example, a physical parameter includes sound, pH, temperature, or pressure. Parametric sensor may include diodes, capacitive pressure sensors, and microphones. Other parametric sensors as known in the prior art could be adapted for use in the device.

The device 35 may also have a second light source 5. The second light source 5 is configured to illuminate a region of the environment external to the housing—a second “field-of-view.” The second light source 5 may be a light-emitting diode (“LED”). The second light source 5 may be located within the housing 102 such that the housing 102 protects the second light source 5 from degradation by alimentary fluid. In such embodiments, the housing 102, or at least a portion of the housing 102, may be transmissive so that the light from the first light source 4 can pass through the housing 102. The second light source 5 may be configured to illuminate a field-of-view at the trailing end of the device 35 (when the device 35 is configured to have ends—e.g., a capsule shape). Alternatively, the second light source 5 may be configured to illuminate a field-of-view at a leading end of the device 35, or a field-a-view at a side of the device 35. The field-of-view may be wide or narrow as suited to the purpose of the device 35. The illumination may be of any brightness and color temperature as suited to the purpose of the device 35. The second light source 5 and first light source 4 can be used in tandem to provide additional illumination to the environment outside the capsule.

In a further embodiment, the first image capture device 2 and the second image capture device 3 are in electronic communication with the analyte sensor 11. The first image capture device 2 and second capture image device 3 capture images based on a signal from the analyte sensor 11. The signal could include an absence, presence, alteration in value, or lack of alteration in value of an analyte. The signal can direct one or both of the image capture devices 2, 3 to capture images. The signal can also direct the image capture devices 2, 3 to capture video. In another embodiment, parametric sensors capture data based on the signal from the analyte sensor.

In another embodiment, the device 35 further comprises a receiver in electronic communication with the first image capture device 2 and the second image capture device 3. The first image capture device 2 and second capture image device 3 capture images based on a signal from the receiver. The signal can also direct the image capture devices 2, 3 to capture video. In another embodiment, parametric sensors capture data based on the signal from the analyte sensor 11.

The device 35 may further comprise a 3-axis accelerometer 14. The accelerometer 14 can be used to measure the magnitude of acceleration and direction of each peristaltic contraction. The information from the accelerometer 14 can be used to determine the location of the capsule within the alimentary canal. The accelerometer 14 can also be used to determine the rate of movement of the capsule. In one example, the accelerometer 14 can be used to measure capsule motion. The physical parameters captured by the accelerometer 14 can be used to compute distance travelled over time and assist with location and tracking of the capsule.

The invention may also be embodied as a method of repeatedly determining an analyte concentration in a fluid. A device configured to be disposed in the fluid is provided 71. The device comprises a sensor substance 30 in a sol-gel material such that the sensor substance 30 reversibly interacts with an analyte of interest. The sensor substance 30 emits electromagnetic energy when the analyte of interest is in contact with the sensor substance 30 and electromagnetic excitation energy is received by the sensor substance 30. The device further comprises an electromagnetic energy source capable of emitting light, and a detector 8 configured to detect electromagnetic energy emitted by the sensor substance. The device may be the device 35 as described herein. The device is moved 73 through at least a portion of the fluid. For example, the device can be moved through peristaltic contractions. The sensor substance is exposed 75 to the fluid. The device may be configured to permit fluid to interact with the sensor substance 30. The detector 8 is used 77 to make at least one measurement of a property of the electromagnetic energy emitted by the sensor substance. For example, the property could be wavelength, intensity, phase, or any other measureable property of an electromagnetic wave. A processor 12 is used to determine 79 at least one analyte concentration value of the fluid based on the plurality of measured properties. In an embodiment, each measured property corresponds to a separate analyte concentration value. A trigger signal is generated 87 for controlling the operation of the device. The trigger signal may be configured based on the determined analyte concentration values. In an exemplary embodiment, the trigger signal can be used to capture images, physical measurements, or control the operation of individual subsystems. One such purpose of controlling individual subsystems is to reduce power consumption by powering down unnecessary portions of the device.

In another embodiment, measurements of emitted electromagnetic energy are made continuously. For example, the detector 8 could capture a plurality of measurements over time to detect changes in the environment outside of the capsule. The measurements may also be taken at regular intervals over time, or in a predetermined pattern. The measurements may also be taken at a predetermined time after the capsule is activated.

The method may further comprise measurement 83 of physical parameters of an environment external to the device by way of a parametric sensor. The physical parameter measurements can be taken both synchronously or asynchronously with the measurements from the analyte sensor. One or more parameters can be measured depending on the configuration of the device.

The method may also comprise transmitting 85 the determined analyte concentration value by way of a transmitter. The analyte concentration values can be transmitted in real-time or at regular intervals. The values may also be transmitted in a single burst. Physical parameter measurements may also be transmitted by way of the same transmitter. The values can be transmitted using a variety of protocols suitable to the purpose of the device.

The method may also comprise receiving 85 a control signal at the device from a remote transmitter. The control signal may contain instructions related to the operation of the device. For example, the control signal could be used to instruct the device to capture an image, start recording video, make physical or analyte measurements, and activate or deactivate device subsystems.

The invention could also be embodied as a method of determining a location of an ingestible device within an alimentary canal. The device would comprise an analyte sensor as described herein. The analyte sensor is used 91 to determine a concentration of an analyte in a fluid proximate to the device. In an embodiment, the analyte sensor would make multiple measurements at different points in the alimentary canal. The location of the ingestible device within the alimentary canal is calculated 99 based on the determined analyte concentration.

The method can further comprise using an accelerometer equipped device to determine a first acceleration 93 of the device. The accelerometer is used to determine a second acceleration of the device. The second acceleration determination is made after the first acceleration. In addition, the determination of the second acceleration corresponds to a determined analyte concentration. For example, the second acceleration 95 determination may be made at a substantially similar time as the analyte concentration, at a predetermined time before the analyte concentration measurement, or at a predetermined time after the analyte concentration. A relative position of the device is determined based on the first and second acceleration 97. The relative position of the device is used in the calculation of the device location in the alimentary canal.

The invention can also be embodied as a method of locating a region of interest within an alimentary canal using an ingestible device. In this method, the ingestible device has an analyte sensor and an accelerometer. The method comprises using 101 the analyte sensor to determine a concentration of an analyte in a fluid proximate to the device. The analyte sensor sends 103 a signal when the analyte sensor senses a concentration of the analyte greater than a pre-determined threshold value, and the pre-determined threshold value corresponding to a region of interest. The accelerometer and the analyte sensor are used to calculate the location of the device within the alimentary canal to determine 105 the location of the region of interest.

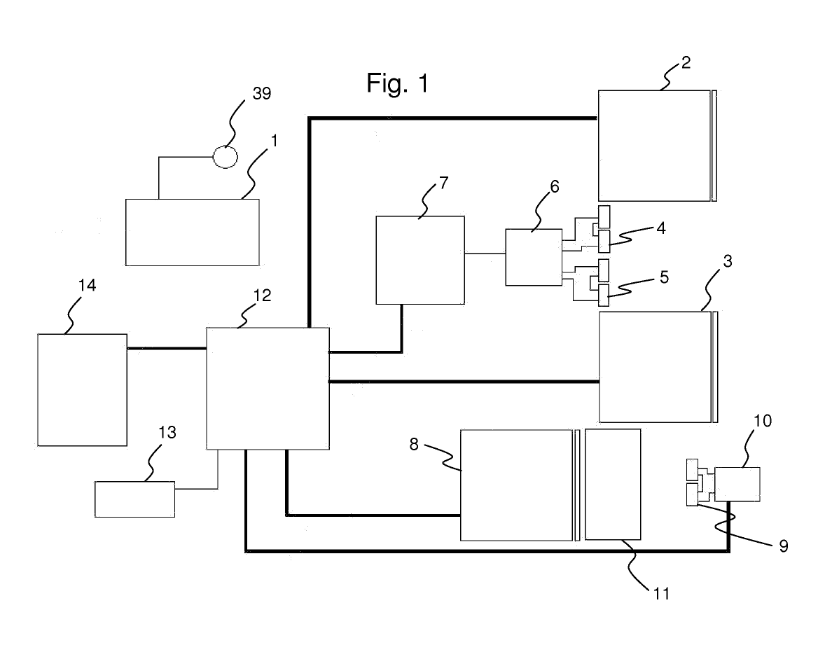

FIG. 1 illustrates the main electronic components in an exemplary embodiment of the device and their electrical interconnections as a generalized functional block diagram. Discrete components supporting each of the major function blocks are not illustrated.

The electronic components used within the capsule comprise at least one image capture device. In this exemplary embodiment, color, video-capable complementary metal oxide semiconductor (CMOS) cameras 2 and 3 are provided. For example, the cameras 2, 3 can be approximately 2.135 mm×2.265 mm in size and provide 1/18″ NTSC Video at 320×240 resolution. It an embodiment, the cameras are mounted at opposing ends of the device, for example, the front and rear ends of a capsule. Camera activity may be event driven to reduce power consumption and to ensure that large amounts of image data do not have to be reviewed to locate an area of the gastrointestinal tract of interest. A clinician monitoring the capsule transit and sensed data outputs can activate the camera 2, 3. Also, the capsule's internal program can activate a camera 2, 3 if a sensed data parameter exceeds a preprogrammed threshold. Likewise, the capsule's internal program can deactivate a camera 2, 3 if a sensed data parameter returns to a nominal, programmed threshold. Two or more cameras may be used because the analyte sensors 30 have a finite response time and capsule transit throughout the gastrointestinal tract is continuous. Therefore, it is possible that the capsule could move beyond the area of the gastrointestinal tract that triggers camera activation before the front camera is activated. In this case the rear camera would capture the area of interest. The cameras 2, 3 are capable of operation in both still frame and continuous video modes. One example of still frame operation (single image) could be triggered when a hemoglobin sensor exceeds a predetermined threshold indicating the presence of blood in the gastrointestinal tract. In another example, a continuous video mode may prove useful to observe peristaltic activity in a section of the gastrointestinal tract. Each of the components in FIG. 1 are energized through power bus 39.

In order to provide adequate illumination for the camera 2, 3 a light source 4, 5 is used in conjunction with the camera. In an embodiment, white Light Emitting Diodes (“LEDs”) are used. A microprocessor 12, a LED circuit 7, and a flash power switch 6 may control the operational mode and synchronization of the LEDs 4, 5 to the camera 2, 3. The LED circuit 7 can provides a high power short duration pulse to flash the camera LEDs 4, 5 in the camera still frame mode. The circuit 7 may also provide continuous, lower power, short duration pulses synchronized to the cameras frame rate for camera operation in continuous video mode. The flash power switch 6 selects the set of active camera LEDs, for example, front or rear. In an embodiment, the camera LED synchronization, mode, image storage and active LED pair is controlled by the microprocessor 12.

In one exemplary embodiment, chemical sensing of analytes contained within the gastrointestinal tract fluid is accomplished by a system comprising LED driver 10, Sensor LEDs 9, sensor cell 11, and CMOS detector array 8, all of which are controlled by microprocessor 12. Microprocessor 12 will initiate measurement at a preprogrammed sampling rate or by an external trigger. The sampling rate can also be modified, increased or decreased, due to a previously sensed parameter threshold-crossing event. The LED driver 10 is activated at each sampling interval to illuminate the sensor LEDs 9, which provide focused optical radiation to the input side of the sensor cell 11. Sensor cell 11 contains active xerogel-based, analyte-interactive material and a filter 31 in contact with the gastrointestinal tract fluid. CMOS detector array 8 monitors the optical radiation from the plurality of xerogel sites and detects an active site. In an embodiment the detector array 8 is 1.815 mm×1.815 mm with 1/10″ analog output at a 400×400 resolution. The level of activation, for example the amount of optical radiation, is passed to microprocessor 12 as an analog value. The value may be digitized, stored, and further processed to determine if event initiation as described above is warranted.

The capsule may also use the output of an accelerometer 14, such as a 3-axis accelerometer, to measure the magnitude of acceleration and direction of each peristaltic contraction in the gastrointestinal tract. In an embodiment, this information and the sensed parameter data is combined to determine the location of the capsule within the gastrointestinal tract, thereby defining the rate of movement. This information may be useful as to monitoring the function of the gastrointestinal tract peristaltic activity. For example, the capsule may rotate about its central axis during transit. This means that the predominant axis of movement from the accelerometer will also rotate for each axis measurement. This problem is addressed by using an amplitude comparison algorithm of current and historic accelerometer output measurements. Digitization, processing of digitized data, data storage, and measurement interval for the accelerometer are controlled by the microprocessor 12.

In a further embodiment, microprocessor 12 controls all functions of the capsule including data storage, data transmission, command reception, camera control, CMOS sensor array operation, and accelerometer operation. The microprocessor 12 also controls battery power conservation. Internally, the microprocessor 12 offers a variety of low power operation modes that are used between active measurement and transmission periods. Power to each functional module is controlled by the microprocessor 12 turning off each system once it has completed its task. In this way the average power consumption of the capsule is minimized. This microprocessor 12 may also contain a full duplex, software radio transceiver capable of transmitting collected data and video. For example, the microprocessor 12 may transmit data if its memory is full. The microprocessor 12 will also accept external commands for control of the capsule subsystems. The transceiver may have an antenna 13. The antenna 13 may be a fractional wavelength pattern etched on a thin sheet of KAPTON®. This embodiment provides a flexible assembly that can be wrapped around the capsule battery 1 using the battery outer metallic foil shell as an active ground plane. Antenna design is modeled after the entire assembly, including the battery outer shell, to optimize RF performance. The radio is implemented using a series of programmable registers which allows software tuning of performance and offers 256 digital transmission/reception channels. Alternatively, the transceiver could be implemented using an 802.11a compatible device or a BLUETOOTH® module. Both options are available in packages with or without embedded antennas in such a form factor that would be suitable for a capsule. The use of an 802.11a or a BLUETOOTH® enabled transceiver allows capsule data and image monitoring using a smartphone with BLUETOOTH® capability or a custom smartphone application. This example would also allow for monitoring the device using an 802.xx enabled device such as a laptop computer or Personal Data Assistant. It is advantageous to both patients and clinicians when a dedicated receiving device is not required. Not only does this reduce cost and complexity, but allows for use of the device without direct medical supervision. Other radio configurations may be used and are within the scope of this disclosure.

In an embodiment of the invention, the battery 1 is comprised of a custom fabricated cylindrical form having an outside diameter no larger than 8 mm and a length of 8 mm.

The battery is rechargeable, using secondary chemistry, and has terminals on the side of the assembly. The battery may also provide 3.4-3.7V to the internal circuitry. The battery 1 outer casing comprises metallic foil, and the battery has a power density greater than 170 mAH/g. The battery may be, for example, a lithium iron phosphate battery or tantalum-based battery. The battery is charged prior to use. Capsule charging can be initiated by piercing the capsule's compliant outer shell with pointed charging terminals aligned with the battery terminal locations of the capsule. The capsule battery terminals are covered by a self-healing silicone rubber compound that maintains the capsule's outer seal when the charging terminals are removed once charging is completed. A series of low noise regulators with on/off control capability are employed to produce “clean”, well regulated power for each function module illustrated in FIG. 1. The microprocessor 12 controls each of these regulators in order to switch power to each function module as required. This approach maximizes battery life. In another embodiment of the invention, the power source is comprised of one or two lithium coin cells that are not rechargeable (e.g. primary chemistry batteries). This type of cell has a very low self-discharge rate enabling a long shelf life. In this embodiment, power to the device is enabled using a switch located behind an elastomeric seal that is mechanically activated by pushing a pin through the self sealing elastomeric membrane.

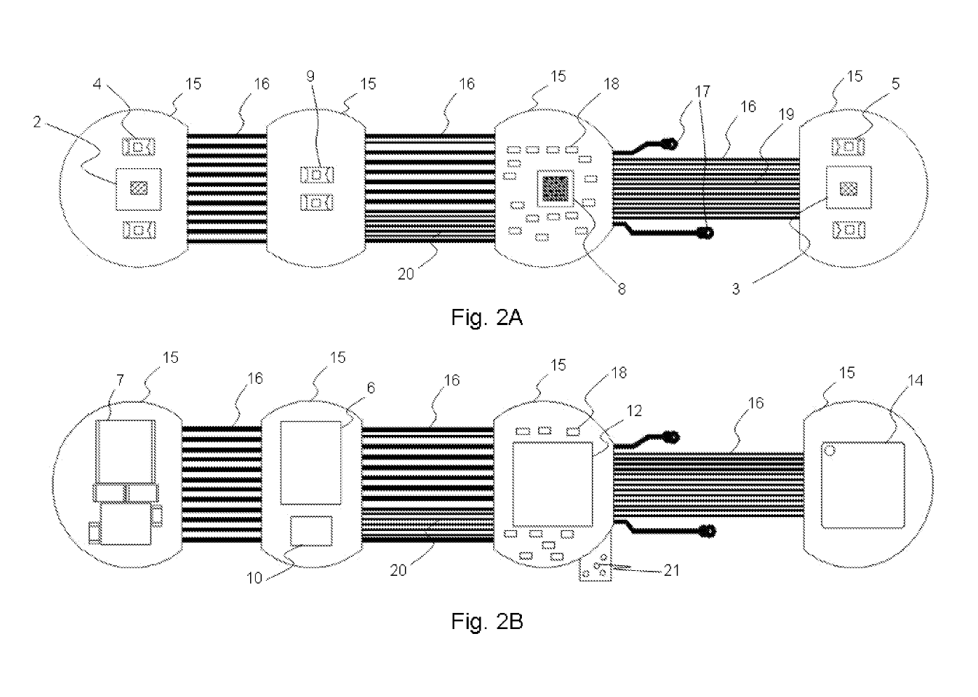

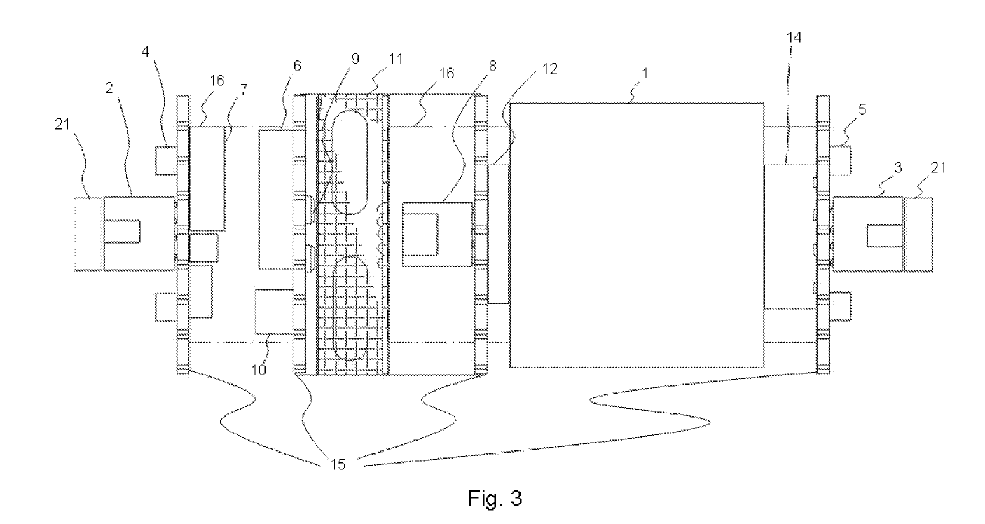

FIGS. 2a and 2b illustrate an assembly method for the capsule's electronic systems. The assembly comprises a series of rigid substrates 15 to which the components in raw integrated circuit die form, or when space allows, chip-scale packages are attached to both the top and bottom sides of the substrate. This method produces a rigid flex assembly optimized for production. In this embodiment, the substrates 15 are comprised of 3 layers of FR4 Printed circuit material having a combined thickness of approximately 0.35 mm. The substrates 15 have the necessary conductive paths supporting the circuitry connections and attachment footprints for the integrated circuits and discrete components. The substrates are interconnected using flexible, insulated fine pitch flat cable assemblies 16 which are embedded into the substrates 15, thus forming connections through the substrate's middle layer to the top and bottom layers and components. This arrangement provides an assembly 16 that can be populated with components using automated assembly techniques when it is in flat form as illustrated in FIGS. 2a and 2b but can be folded into a capsule shaped molding form or for insertion into a pre-molded outer shell.

In an embodiment, the CMOS cameras 2, 3 and lighting devices 4, 5 are located on the topside of the end substrates 15 as illustrated in FIG. 2a. The topside of the two middle substrates 15 contains the sensor LEDs 9 and the CMOS image sensor 8. The sensor cell 11 is mounted in the area 20 between the two middle substrates. The battery is mounted in the area 19 between the rear CMOS camera 3, substrate 15, and/or the CMOS image sensor 8, and substrate 15. Battery terminals 17 are backed with a thin sheet of brass to prevent a puncturing of the battery 1 case when the terminals 17 are covered with silicone rubber as described above. The location of each of the required discrete components 18 can be determined through computerized optimization of the printed wiring for each substrate. Each substrate 15 can contain these components in surface mount technology package form.

FIG. 2b illustrates the component layout in a embodiment for the bottom side of the assembly. The first substrate 15 (starting on the left side of FIG. 2b) contains the camera LED circuit 7, followed by a substrate 15 containing the flash power switch 6 and Sensor LED driver 10. The third substrate 15 contains the microprocessor 12 and transceiver integrated circuit in die form wire bonded to the substrate 15 as well as the radio frequency matching components and antenna 13 connections. A breakaway tab is also attached to this substrate 15. This tab contains connections to a USB interface attached to the microprocessor 12. This interface can be used for initial device setup, radio programming, and diagnostic testing during production. After testing is complete, the tab is removed from the substrate 15. The fourth substrate 15 (on the right side of FIG. 2b) contains the 3-axis accelerometer 14 integrated circuit. Body core temperature can be measured using a diode based temperature sensor located within the die for microprocessor 12. In this embodiment, thermal lag measurement and calibration is used to determine the exact time and location of each temperature measurement.

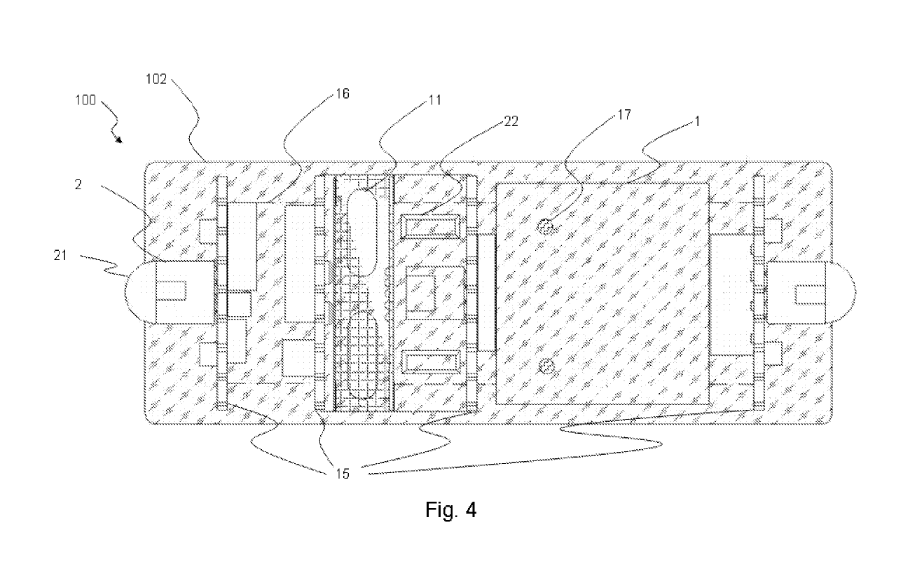

In an embodiment, folding of the rigid flex assembly can be accomplished using a mold with features for proper alignment of the substrates 15 if the assembly is to be encapsulated using a 2 part room temperature medium viscosity material. Alternatively, a fixture can be used that will also have these alignment features and allow adhesive fixing of the substrates 15, sensing cell 11 and battery 1 in the final configuration for insertion into a plastic shell. FIG. 4 illustrates the folded form of the assembly.

The locations of the battery 1 and sensing cell 11 are illustrated in FIG. 4. Each of the CMOS cameras 2 and 3 require a short focal length lens 21 to be attached. The center of each of these lenses 21 forms the boundary lines for encapsulation of the assembly. Once folded, the flex interconnect cable assemblies 16 fall alternately on the front side and back side of the assembly. The receiving antenna 13 for the capsules radio frequency transceiver (located in microprocessor 12) is wrapped around the battery, and is not shown in this illustration.

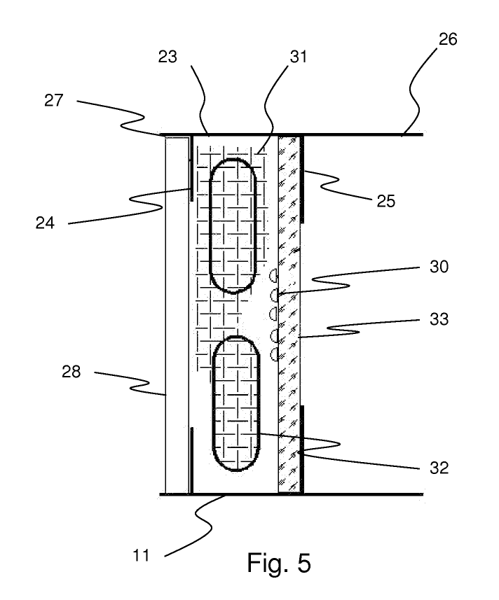

FIG. 5, illustrates another embodiment featuring outer geometry for the capsule after encapsulation. Alignment features 22 have been added to the encapsulation mold to provide for proper alignment of the capsule within a charging/activation stand. These features consist of shallow wells molded into the assembly designed to mate with a corresponding geometrical post within the charge/activation stand and capsule holding assembly. The geometric posts will insure that battery terminals 17 will mate properly with the correct polarity of the pointed terminals within the charge/activation stand. One benefit of this design is apparent if the capsule is not inserted properly into the charge/activation stand. Then, the capsule will sit too high in the stand to allow the pointed terminals to penetrate the capsules shell, preventing damage to the battery.

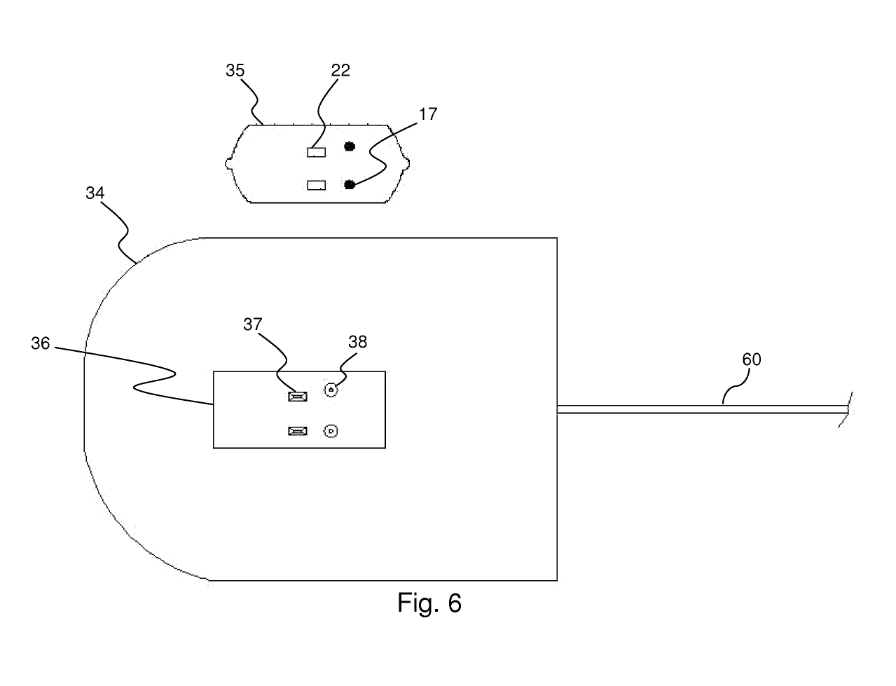

FIG. 6, illustrates the configuration of the sensing cell 11. The sensing cell 11 is comprised of an injection molded circular outer shell 23 with two internal lips 24 and 25. In an exemplary embodiment, the front lip 24 has an opening diameter of 5 mm while the rear lip 25 has an opening diameter of 4 mm. The sensor cell 11 may have a thickness of 2.5 mm. These features are designed to provide attachment and sealing points for optically clear windows 28 and 33. The larger front lip opening 24 provides a simple method for assembly of the cell windows from the front of the device 24 using a smaller diameter window 33 for the rear of the cell. The diameter of the front window 28 is 6 mm while the diameter of the rear window 33 is 5 mm. Both windows can be 0.2 mm thick N-BK7 glass or fabricated from clear plastic or quartz designed to allow passage of optical wavelengths used by the cell. The front and rear windows 28 and 33 form a liquid tight seal between the sensing cell 11 and the substrates 15 of the rigid flex assembly. The length of the outer shell 23 is such that a lip extends beyond windows 28 and 33 to form an alignment feature for the rigid flex assembly substrates containing the sensor LEDs 9 and the CMOS image sensor 8. This feature is illustrated as callouts 26 and 27. The rear window 33 of the cell may face the CMOS image sensor 8 and will preferably have the desired optical filter deposited or attached to the window 33. The sol-gel material based sensing sites 30 will also be printed to the inside surface of this window. The sensing cell outer shell 23 also has elongate slots 32 molded into the circumference of the shell providing a path for fluid. In an embodiment, the cell is filled with 0.9% normal saline and the elongated slots 32 are covered with a semi-permeable membrane 31 that allows fluid flow/exchange to and from the cell.

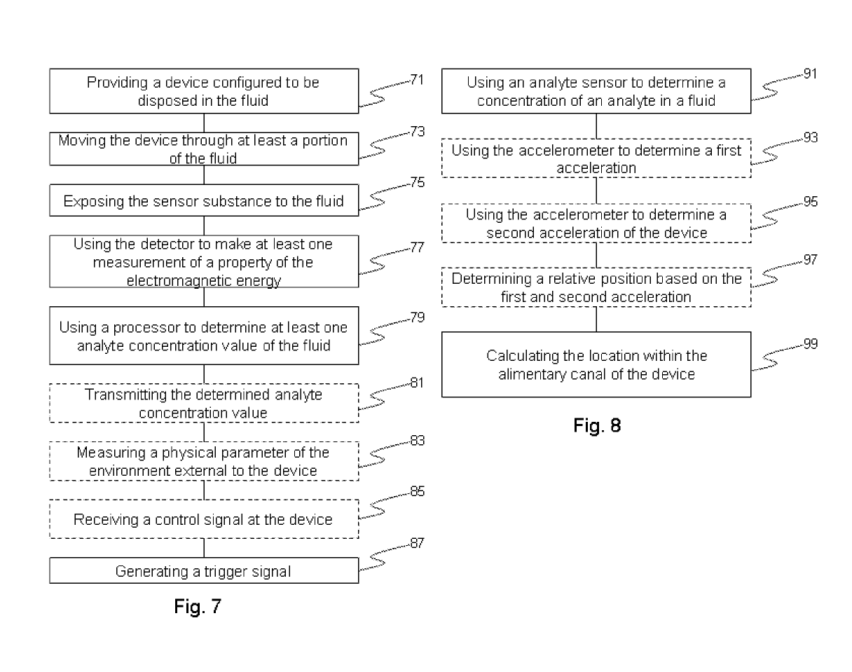

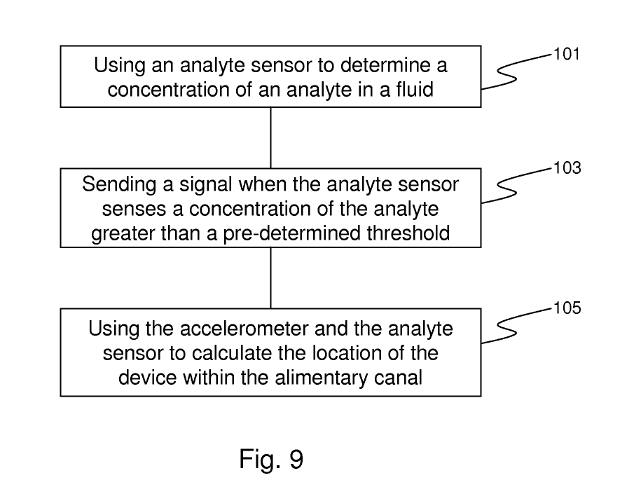

FIG. 7 illustrates an embodiment of the charge/activation stand 34 for the capsule. The charge/activation stand may comprise a plastic shell containing a capsule fixing well 36 designed to hold capsule 35 for activation and charging. Two post-like features 37 designed to insure proper alignment of capsule 35 in the stand by mechanically mating with alignment wells 22. Two pointed battery charging pins 38 are designed to pierce the self-healing silicon rubber caps on the capsules battery terminals 17. The charge/activation stand 34 may also include a radio transceiver designed to monitor and control the capsule 35, a USB to PC interface 60, a USB powered battery charging circuit, and a hinged lid designed to hold the capsule in position during charging.

Embodiments of the present invention can provide an ingestible capsule for use in continuous measurement of concentrations of substances in the fluids of the gastrointestinal tract of an animal. The capsule can include an electric power source, a radio signal transmitter in enabling circuitry with the power source suitable for transmitting a radio signal which contains concentration information from the sensing composite. In some embodiments, a radio receiver provides the device with the ability to accept external commands via radio transmission, and the external control of all data storage, transmitting, collection methods and data sampling rates in real-time. The external control can provide the device status, or change transmission modes on request. A detection module suitable for measuring luminescence intensity can be associated with an imaging device to capture a visual depiction of the local area of interest in the alimentary canal. An optical tracking mechanism based on light reflectance and capsule movement can also be used. The capsule and its components can be encased in a non-digestible outer shell that is configured to pass through the alimentary canal.

The electric power source for the capsule can comprise one or more batteries, and the electric power source can be a thin film rechargeable battery. The transmitter can emit a radio signal that is detectable exterior to the outer shell of the capsule. The power source consumption of the capsule can be controlled by “smart software” employing numerous power modes to extend battery life. An internal non-volatile memory can be employed for the storage of any sensed data.

The receiver can provide direct control of transmission modes including burst type transmission to reduce power consumption. The receiver can also allow external commands to request transmission of capsule battery, sensor, and memory status. The transmitter and receiver are also capable of operation over 256 RF digital channels providing the capability of monitoring multiple capsules operating in close proximity without interference. The capsule can also have a transmitter and receiver that employ a fractional wavelength antenna integrated onto the battery case.

The capsule can use an analyte sensor that functions continuously to detect chemicals in the fluids of the intestinal tract for the entire time it is present in the intestinal tract. The capsule can incorporate an optical device of forming a digital image of a part of the internal alimentary canal adjacent to the capsule, and transmit this image to the external receiver. The image can be taken when chemical sensing detects an area of interest, in one example the detection of bleeding at a specific location, or some other problem.

The analyte sensor can incorporate a luminescence detection of analytes. More particularly, the present disclosure provides a device wherein the electromagnetic radiation generator provides a substrate for chemical sensors, where the spectroscopic properties of the chemical sensors are modified upon contacting an analyte. The disclosed method can include the selective and simultaneous detection and quantification of multiple analytes, and a method of making the device useful in the intestinal tract of an animal. The capsule can have one or more chemical sensors for interacting selectively with a particular analyte in a sample. In the absence of the analyte, the chemical sensor displays certain baseline spectroscopic properties characteristic of the sensor. However, when the analyte is present in the sample, the spectroscopic properties of the chemical sensor are modified. Detection and quantification of the analyte are based on a comparison of the modified properties and the baseline properties and the use of standard calibration methods that are well known to those skilled in the art of analytical chemistry.

In some aspects the presently disclosed capsule has a chemical sensor that comprises a reporter molecule whose optical properties are modified in the presence of an analyte.

The properties of the sensor element may be directly modified upon its interaction with the analyte. Alternatively, the reporter molecule may be attached to a template material having a specific affinity for the analyte, in which case, the optical properties of the reporter molecule are modified upon the interaction of the template material with the analyte. Thus, by the term “spectroscopic properties of the chemical sensor” or “chemical sensor's spectroscopic properties” it is meant the spectroscopic properties of the reporter molecule and vice versa. These properties may be optical in nature when the emitted electromagnetic radiation is within the visible spectrum i.e., between about 400 nm to about 800 nm. As an example, if the chemical sensor is a site selectively templated and tagged xerogel (SSTTX) or a protein imprinted xerogel with integrated emission site (PIXIES), the reporter molecule is one or more luminescent reporter molecules within a molecularly templated xerogel and the analyte affinity is afforded by the template sites within the xerogel. In another example, where the chemical sensor is a luminescent ruthenium dye (tris(4,7-diphenyl-1,10-phenanthroline)ruthenium(II), ([Ru(dpp) 3]2+), the reporter molecule ([Ru(dpp h]2+) provides an analyte-dependent response directly.

The types of analytes that may be detected include both liquid and gaseous materials.

These include CO2, O2, cytokines, interleukins, incretins, carbohydrates, hormones, hemoglobin, proteins, peptides, nutrient substances, vitamins, pesticides, drugs, herbicides, anions, cations, antigens, oligonucleotides, and haptens. Further, the present disclosure can chemically indicate the pH and salinity of a sample. In addition, chemical sensors are available and can be used in the present disclosure to detect the presence of organic molecules such as polycyclic aromatic hydrocarbons, glucose, cholesterol, amino acids, and peptides. Further, the presently disclosed capsule can detect the presence of bacteria and viruses of both normal and pathogenic nature. There are many more substances which can be detected, and the foregoing list is not to be considered exhaustive, but instead is merely representative.

The electromagnetic radiation emitted by the chemical sensor may be detected by any suitable method known in the art. A general configuration in the figures shows a detecting device in combination with a receiving and interpreting system. The receiving and interpreting system has a receiver to receive electromagnetic radiation transmitted or emitted by the chemical sensor(s) and convert the optical signal into an electrical signal, and an interpreter to interpret the received electrical signal. In an embodiment, the receiver is a CMOS based array with a filter preceding the receiving surface on the CMOS array. The receiver may have a camera for recording images. The interpreter can include a controller and a computer having software running thereon. The receiving surface is connected to the controller. One or more filters may be placed between the substrate and the receiving surface. The filter selectively passes desired wavelengths of the electromagnetic radiation moving from the detecting device toward the receiving surface and blocks undesired wavelengths. An example of a filter which can be used to practice the present disclosure is model number XF 3000-38 manufactured by Omega Optical of Brattleboro, Vt. This particular filter passes electromagnetic radiation above approximately 515 nm and strongly attenuates electromagnetic radiation below approximately 515 nm. Other filters or filter combinations are possible depending on the generator wavelength and the particulars associated with a given sensor.

The sample to be analyzed may be continuously exposed to the chemical sensor(s), and the receiver components are placed in the proper position to permit the receiving and interpreting system to receive radiation from the chemical sensors. The electromagnetic information collected during operation may be digitized to provide input to a digital memory during sensing, and sent to a receiving device over wireless communications at time intervals.

The capsule may use one or more SSTTX- or PIXIES-based sensors. The luminescence output from these types of sensors is stable for many days under constant excitation. Thus, this demonstrates that using the method of the present disclosure, the chemical sensor platform is sufficiently stable to be used for detection and quantification of analytes in the intestinal tract. The chemical sensing of the capsule of the present disclosure can use the analysis specifications and methods contained in U.S. Pat. Nos. 6,241,948 and 6,589,438, which are incorporated herein by reference, and apply them to a non-digestible sensing capsule designed for use in the intestinal tract. The present disclosure provides a detecting device wherein the chemical sensor can be placed in contact with the electromagnetic radiation generator that excites the luminescent reporter molecules within the sensors, making the device compact and suitable for incorporation in the capsule of the present disclosure. Furthermore, the electromagnetic radiation used in some embodiments of the present disclosure is not reflected, filtered, or transmitted over a long distance prior to reaching the chemical sensors. In addition, the detecting device according to the present disclosure can be made relatively inexpensively and readily mass produced.

An optical fiber brush may be employed to clear the active surface of the analyte sensor between measurements. The optically clear brush may be connected to an optically clear disk and driven by a small electrical motor to both clear the analyte sensor and draw in fresh fluids of the intestinal tract. The optically clear brush may also be surrounded by an electrically active polymer sleeve which when electrically charged changes shape and functions to both clear the analyte sensor and draw in fresh fluids of the intestinal tract. The xerogel-based sensor platform described may continuously detect one or more analyte molecules in intestinal fluids in relation to concentration, and wherein the luminescent signal emitted by the sensors occurs in strength proportional to analyte concentration in intestinal tract fluids as the ingested capsule travels the length of the intestinal tract.

The xerogel-based sensor platform associates and dissociates reversibly to its analyte molecule, enabling continuous signal emulation in proportion to changing concentration of the analyte molecules in intestinal fluids. The molecular analysis substance is any other composite or molecule which binds reversibly to its analyte molecule, enabling continuous luminescence signal emulation in relation to changing concentration of the analyte molecules in intestinal fluids.

In some aspects the disclosed capsule device is for repeated detection of the concentration of at least one analyte in a sample that comprises: an electromagnetic radiation generating source having at least one SSTTX- or PIXIES-based sensor formed directly on, or on a substrate that is in turn in close proximity to the electromagnetic radiation generating source such that the analyte containing stream can come into contact with the sensor, and the spectroscopic properties of the chemical sensor are modified in the presence of the analyte. The electromagnetic radiation generating source may be a light emitting diode.

The sensing system can further comprise a receiving and interpreting system having an electromagnetic radiation receiver to receive electromagnetic radiation emitted by the chemical sensors, which has the capability to interpret the received electromagnetic radiation. The receiver can further include a filter for selectively passing electromagnetic radiation. The receiver can include a CMOS based array, a charge coupled device, and a lens for focusing the electromagnetic radiation on the charge coupled device. In some aspects the receiver also includes an opaque shield above the lens for focusing the electromagnetic radiation on a CMOS based array device. The interpreter can include a storage device for storing the digitized data output from the CMOS array.

The sensing system can also have a holding substrate for holding the chemical sensors in optical alignment with the electromagnetic radiation generating source, one or more filters, and a receiver. The holding substrate can be a xerogel or other material (e.g., glass, plastic) that is not degraded in the fluids of the intestinal tract. The holding material can also be comprised of tetramethylorthosilane. In some aspects the chemical sensor(s) is(are) comprised of a reporter molecule and an analyte-responsive template (e.g., SSTTX or PIXIES) having a specific affinity for the analyte. The reporter molecule can be selected from the group comprising fluorophore, phosphore and chromophore.

The distance travelled by the capsule within the intestine may be determined by optical recording of distance travelled in a forward direction over the time the capsule resides within the intestinal tract. As can be seen from the aforesaid recitation, the process of the disclosed capsule generally comprises obtaining directional data from the passage of a signal transmitting capsule through an alimentary canal to create a precise map of the routing of the capsule to a precise location in the canal.

The distance travelled by the capsule within the intestine may also be determined by recording of distance travelled over the time the capsule resides within the intestinal tract using an accelerometer. As can be seen from the aforesaid recitation, the process of the disclosure generally comprises obtaining directional data from the passage of a signal transmitting capsule through an alimentary canal to create a precise map of the routing of the capsule to a precise location in the canal. The capsule distance traveled as defined above can be used in combination with the sensed physiologic data and/or chemistry data to enhance the map of capsule routing and provide localization data for the capsule in real time.

The LED of the capsule can project light into the field of view of the lens via a prism. The prism enables the LED to be set horizontally, which conforms to the size dimensions of ingestible capsules and reduces optical losses. The prism and lens may form part of a single molding in an embodiment.

The capsule may in some embodiments include a light source for emitting light in the optical detection of distance travelled per unit of time, an irradiating lens for irradiating the light emitted from the light source to a surface, a light-receiving lens for condensing light irregularly reflected upon the surface and the light emitted from the light source and irradiated through the irradiating lens, a total reflection prism that can allow the light emitted from the light source and irradiated through the irradiating lens to be located on a path where totally reflected light upon the surface advances (after transmitting through a transparent material such as a glass, and reflecting incident light through a reflecting surface to be condensed on the light-receiving lens), and an optical sensor for sensing the light condensed through the light-receiving lens.

The capsule can contain a remote actuatable storage reservoir which comprises a receiver for receiving a radio signal from a remote transmitter positioned exterior of the outer shell of the capsule.

The storage reservoir may be actuated by an activator, which comprises an initiator coil, enabling circuitry with the power source, that is characterized by emitting a detectable resonate frequency when the activator is activated. A sampling storage device and a remote actuatable sampling device can also be included. The capsule can signal a remote receiver as it progresses through the alimentary tract and upon reaching a specified site is remotely triggered to capture a fluid sample in the alimentary canal. The results of continuous analyte detection can trigger the remote actuatable sampling device. The storage compartment reservoir can be closed by an actuator controlled by a transmitter positioned exterior of the outer shell of the capsule. Intestinal fluid from the storage reservoir can be recovered for chemical assay upon collection of the capsule expulsed, and PCR assay can be conducted upon collection of the fluid. The storage reservoir can be recovered for assay of analytes. The outer shell of the capsule can be made of a polycarbonate. The capsule can also provide chemical sensing on a continuous basis in any fluid containing environment.

The enabling circuitry can comprise a switching device and the said enabling circuitry comprises a polymeric seal about conductive terminals to charge (activate) the battery prior to use.

In an embodiment the disclosure provides an ingestible capsule for delivery of a medicament to the alimentary canal comprising, a non-digestible outer shell; an electric power source; a radio signal transmitter in enabling circuitry with the power source suitable for transmitting a radio signal, the location from which it emanates being determined from distance travelled measured optically or chemically, and, a remote actuatable intestinal fluid capture sampling and releasing device. The capsule can utilize a quantified DC voltage signal that is digitized to provide input to a computer. The capsule can also use a time multiplexed output of the multiple sensors is converted to an intermediate frequency signal, quantified as a DC voltage signal and digitized to provide input to a computer. In some aspects, transmitted signals are received exterior of the animal body digitized and provided to a computer. The digitized information has parameters comprising one of rate of progress of the capsule through the canal, length of time of the capsule in the canal or specific locations in the canal.

The capsule computer can be programmed to scan and compute variations from preprogrammed factors. The computer can be programmed to initiate an actuating signal to the medicament releasing device of the capsule. The actuating signal can be operator controlled. Capture of intestinal fluid sample initiates an indicator signal, by a capture indicator signal device comprised in the capsule, which is detectable exterior of the body of the animal.

Two or more capsules can be used in the alimentary canal to transmit differential signals.

The first ingestible capsule, containing a signal transmitter, is ingested into the alimentary canal; a signal transmitted from that capsule is received exterior of the body and digitized in a computer; and the digitized data from a signal transmitted from the first capsule comprises the pre-established model.

The present disclosure may be embodied as a method for the continuous collection of sensing data in the alimentary canal of an animal and capture of intestinal fluids for remotely triggered collection of sensing data after capsule expulsion. The method can comprise: providing an ingestible capsule containing a radio signal transmitter suitable for determining location of the capsule; ingesting the capsule into the alimentary canal; transmitting a radio signal from the transmitter; receiving the transmitted signal exterior of the body of the animal. The signal can be digitized after reception by multiple antennae and then stored in computer recoverable, time sequence memory. The present disclosure also includes an ingestible capsule for continuous collection of sensing data in the alimentary canal of an animal comprising, a non-digestible outer shell.

The capsule can have an electric power source; a radio signal transmitter in enabling circuitry with the power source suitable for transmitting a radio signal the location from which it emanates as it travels through alimentary canal. The capsule can further include an intestinal fluid capture reservoir compartment comprising a filling membrane, arranged to move in response to generation of the actuator signal. The capsule can further comprise a radio signal receiver. A device for measuring sensing signals from intestinal fluids can also be included in the capsule, where the output is converted to time multiplexed output.

A data receiver can be worn by the patient (e.g. on a belt clip or lanyard); be self-powered (e.g. 5 or more days battery life); having an easy-to-use patient activated “Event” button; where the data is downloaded to a PC through companion docking station; and receives and stores data from capsule. The data receiver can automatically processes data and displays test results such as: smart pill gastric emptying time; anterior duodenal pressure; total transit time; and combined small/large bowel transit time. Complete test results are available for review and analysis in minutes. These functions can be performed in a laboratory, or preferably, on a smartphone device.

Embodiments of the present invention also include, without limitation, the following examples and combinations thereof:

### Example 1

An ingestible capsule for use in continuous sensing in the fluids of the gastrointestinal tract of an animal, comprising, an electric power source, a radio signal transmitter in enabling circuitry with said power source suitable for transmitting a radio signal which contains concentration information from the sensing composite, a radio receiver which provides the device with the ability of accepting external commands via radio transmission, said external control of all data storage, transmitting, collection methods and data sampling rates “on the fly,” said external control to provide device status or change transmission modes on request, a device for initiating digitized still frame or video photos of the alimentary canal, an internal operating program that provides the ability to pre-program events including sensor sampling rate adjustment and CMOS digital camera operation based on sensed data thresholds, an optical detection capability suitable for measurement of luminescence intensity, and an electromechanical tracking mechanism based on light reflectance or sensor fusion with the output of a 3-axis accelerometer, all encased in a non-digestible outer shell that is configured to pass through said alimentary canal.

### Example 2

The capsule of example 1, wherein said electric power source comprises a secondary chemistry cylindrical shape having battery terminals sealed using self healing silicone rubber covers.

### Example 3

The capsule of example 2, wherein said electric power source comprises a thin film rechargeable battery.

### Example 4

The capsule in example 2 wherein said electric power source is comprises a primary chemistry lithium coin type battery cell or cells activated by pushing a pin located behind an elastomeric self healing membrane to complete the battery connection (switch) the device on.

### Example 5

The capsule of example 1, wherein said transmitter emits a radio signal, detectable exterior to said outer shell of said capsule, when enabled by said power source.

### Example 6

The capsule of example 1, wherein said power source consumption is controlled by “smart software” employing numerous power modes of the microprocessor to extend battery life.

### Example 7

The capsule of example 1, wherein said power source consumption is controlled by “smart software” that can enable or disable power to each of the capsules sub-systems as needed to provide the currently requested measurement or data transmission function.

### Example 8

The capsule of example 1, wherein said power source consumption can be externally controlled using the transceiver interface to enable or disable any of the capsules measurement functions.

### Example 9

The capsule of example 1, wherein internal non-volatile memory is used for the storage of sensed data.

### Example 10

The capsule of example 1, wherein said receiver provides direct control of transmission modes including burst type transmission to reduce power consumption.

### Example 11

The capsule of example 1, wherein said receiver allows external commands to request transmission of capsule battery status, sensor and memory status, control of onboard CMOS camera mode and operation, sensor sampling rate and control of optical drive for the sensors.

### Example 12

The capsule of example 1, wherein said transmitter and receiver are capable of operation over 256 RF digital channels, which enables the operation of multiple capsules in close proximity without interference.

### Example 13

The capsule of example 1, wherein said transmitter and receiver use a fractional wavelength antenna integrated onto the battery case.

### Example 14

The capsule of example 1, wherein said transmission and receiver comply with 802.xx or BLUETOOTH® standards allowing monitoring using a “smart phone” or 802.xx enabled device.

### Example 15

The capsule of example 1, that uses an analyte sensor that functions continuously to detect chemicals in the fluids of the animal intestinal tract for the entire time it is present in said intestinal tract.

### Example 16

The capsule of example 1, wherein said analyte sensor uses luminescence detection of analytes. More particularly, the present invention provides a device wherein the spectroscopic properties of the chemical sensors are modified upon contacting an analyte, and the effects of changing concentration are detected as electromagnetic radiation.

### Example 17

The capsule of example 1, which provides a method for the selective and simultaneous detection and quantification of multiple analytes, and a method of enabling this property in the intestinal tract of an animal.

### Example 18

The capsule of example 1, having one or more chemical sensors for interacting selectively with a particular analyte in a sample. In the absence of the analyte, the chemical sensor displays certain baseline spectroscopic properties characteristic of the sensor. However, when the analyte is present in the sample, the spectroscopic properties of the chemical sensor are modified. Detection and quantification of the analyte are based on a comparison of the modified properties and the baseline properties and the use of standard calibration methods that are well known to those skilled in the art of analytical chemistry.

### Example 19

The capsule of example 1, wherein a chemical sensor comprises a reporter molecule whose electromagnetic radiation properties are modified in the presence of an analyte. The properties of the sensor element may be directly modified upon its interaction with the analyte. Alternatively, the reporter molecule may be attached to a templated material having a specific affinity for the analyte, in which case, the electromagnetic radiation properties of the reporter molecule are modified upon the interaction of the templated material with the analyte. Thus, by the term “spectroscopic properties of the chemical sensor” or “chemical sensor's spectroscopic property” is meant the spectroscopic properties of the reporter molecule and vice versa. These properties may be optical in nature when the emitted electromagnetic radiation is within the range of between about 270 to 1000 nm. As an example if the chemical sensor is a site selectively templated and tagged xerogel (SSTTX) or a protein imprinted xerogel with integrated emission site (PIXIES), the reporter molecule is one or more luminescent reporter molecules within a molecularly templated xerogel and the analyte affinity is afforded by the template sites within the xerogel. In another example, where the chemical sensor is a luminescent ruthenium dye (tris(4,7-diphenyl-1,10-phenanthroline)ruthenium(II), ([Ru(dpp)3]2+), the reporter molecule ([Ru(dpp)3]2+) provides an analyte-dependent response directly.

### Example 20

The capsule of example 1, whereby types of analytes that may be detected include both liquid and gaseous analytes. These include CO2, O2, cytokines, interleukins, incretins, carbohydrates, hormones, hemoglobin, proteins, peptides, pesticides, drugs, herbicides, anions, cations, antigens, oligonucleotides, and haptens. Further, the present invention can chemically indicate the pH and salinity of a sample. In addition, chemical sensors are available and can be used in the present invention to detect the presence of organic molecules such as polycyclic aromatic hydrocarbons, glucose, cholesterol, amino acids, and peptides. Further, the present invention can detect the presence of bacteria and viruses of both normal and pathogenic nature. There are many more substances which can be detected, and the foregoing lists are not to be considered exhaustive, but instead is merely representative.

### Example 21

The capsule of example 1, whereby the electromagnetic radiation emitted by the chemical sensor may be detected by any suitable method known in the art. A general configuration shows a detecting device in combination with a receiving and interpreting system. The receiving and interpreting system has a receiver to receive electromagnetic radiation transmitted or emitted by the chemical sensor(s) and convert the optical signal into an electrical signal and an interpreter to interpret the received electrical signal. In an embodiment the receiver is a CMOS based array with a wavelength filter preceding the receiving surface on the CMOS array. The receiver may have a camera for recording images. The interpreter includes a controller and a computer having software running thereon. The receiving surface is connected to the controller.

### Example 22

The capsule of example 1, wherein one or more filters may be placed between the substrate and the receiving surface. The filter selectively passes desired wavelengths of the electromagnetic radiation moving from the detecting device toward the receiving surface and blocks undesired wavelengths. An example of a filter that can be used to practice the present invention is model number XF 3000-38 manufactured by Omega Optical of Brattleboro, Vt. This particular filter passes electromagnetic radiation above approximately 515 nm and strongly attenuates electromagnetic radiation below approximately 515 nm. Other filters or filter combinations are possible depending on the generator wavelength and the particulars associated with a given sensor.

### Example 23

The capsule of example 1, wherein the sample to be analyzed is continuously exposed to the chemical sensor(s), and the receiver components are placed in the proper position to permit the receiving and interpreting system to receive radiation from the chemical sensors.

### Example 24

The capsule of example 1, wherein the electromagnetic information collected during operation is digitized to provide input to a digital memory during sensing, and sent to a receiving device over wireless communications at time intervals.

### Example 25

The capsule of example 1, uses one or more SSTTX- or PIXIES-based sensors. The luminescence output from these types of sensors is stable for many days under constant excitation. Thus, this data demonstrates that using the method of the present invention, the chemical sensor platform is sufficiently stable to be used for detection and quantification of analytes in the intestinal tract.

### Example 26

The capsule of example 1, wherein the chemical sensor can be placed in contact with the electromagnetic radiation generator that excites the luminescent reporter molecules within the sensors, making the device compact and suitable for incorporation in the capsule of example 10. Furthermore, the electromagnetic radiation used in the present example is not reflected, filtered, or transmitted over a long distance prior to reaching the chemical sensors. In addition, the detecting device according to the present invention can be made relatively inexpensively and readily mass produced.

### Example 27

The capsule of example 1, wherein the analyte sensor of example 11 is fabricated as a pre-assembled cell containing xerogel-based sensing sites, wavelength filter, clear sealing windows and alignment features for radiation source and detector.

### Example 28

The capsule of example 1, wherein the chemical sensing cell of example 23 is filled with normal 0.9% saline and an osmotically active substance such as high molecular weight dextran, and covered with a semi-permeable membrane allowing communication between the property to be sensed and the and the chemical sensing sites using osmotic process.

### Example 29

The capsule of example 1, wherein the chemical sensing cell of example 23 wherein the required optical filter is vapor or otherwise deposited to the surface of one of the optically clear seals and the xerogel-based sensing sites are printed directly to this surface.

### Example 30

The capsule of example 26, wherein said xerogel-based sensor platform continuously detect one or more analyte molecules in intestinal fluids in relation to concentration, and wherein the luminescent signal emitted by the sensors occurs in strength proportional to analyte concentration in intestinal tract fluids as the ingested capsule travels the length of the intestinal tract.

### Example 31

The capsule of example 26, wherein said xerogel-based sensor platform associates and dissociates reversibly to its analyte molecule, enabling continuous signal emulation in proportion to changing concentration of said analyte molecules in intestinal fluids.

### Example 32

The capsule of example 26, wherein said molecular analysis substance is any other composite or molecule which binds reversibly to its analyte molecule, enabling continuous luminescence signal emulation in relation to changing concentration of said analytes molecules in intestinal fluids.

### Example 33

A capsule device for repeated detection of the concentration of at least one analyte in a sample, comprising: an electromagnetic radiation generating source having at least one SSTTX- or PIXIES-based sensor formed directly on or on a substrate that is in turn in close proximity to the electromagnetic radiation generating source such that the analyte containing stream can come into contact with the sensor, wherein the spectroscopic properties of the chemical sensor are modified in the presence of the analyte.

### Example 34

The device of example 33, wherein the electromagnetic radiation generating source is a light emitting diode.

### Example 35

The sensing system of example 33, further comprising a receiving and interpreting system having electromagnetic radiation receiver to receive electromagnetic radiation emitted by the chemical sensors, and having a capability to interpret the received electromagnetic radiation.

### Example 36

The sensing system of example 33, wherein the receiver includes a filter for selectively passing electromagnetic radiation.

### Example 37

The sensing system of example 33, wherein the receiver includes a CMOS based array for sensing the electromagnetic radiation passed by the filter of example 32.

### Example 38

The sensing system of example 33, wherein the receiver includes a lens for focusing the electromagnetic radiation on the CMOS.

### Example 39

The sensing system of example 33, wherein the receiver includes an opaque shield above the lens for focusing the electromagnetic radiation on a CMOS based array device.

### Example 40

The sensing system of example 33, wherein the interpreter includes a storage device for storage of digitized data output from the CMOS array.

### Example 41

The sensing system of example 33, further comprising a holding substrate for holding the chemical sensors in optical alignment with the electromagnetic radiation generating source, one or more filters, and a receiver.

### Example 42

The device of example 41, wherein the holding substrate is a xerogel or other material (e.g., glass, plastic) that is not degraded in the fluids of the intestinal tract.

### Example 43

The device of example 42, wherein the holding material is comprised of one or more organosilane-based sol-gel derived xerogels.

### Example 44

The sensing system of example 33, wherein the chemical sensor(s) is(are) comprised of a reporter molecule and an analyte-responsive template (e.g., SSTTX or PIXIES) having affinity for the analyte.

### Example 45

The sensing system of example 33, wherein the reporter molecule is selected from the group consisting of fluorophore, phosphore, and chromophore.

### Example 46

The capsule of example 1, wherein the diode based temperature sensor contained on the integrated circuit die for the microprocessor is calibrated over the core body temperature range and used to measure body core temperature.

### Example 47

The capsule of example 1, wherein the distance travelled and the direction of each movement within the intestine by said capsule is determined by data analysis of the digitized output of a 3-axis accelerometer contained within the capsule.

### Example 48

The capsule of example 1, wherein the processed 3-axis accelerometer output is mathematically analyzed with sensed data such as pH to produce an accurate real time location of the capsule in the intestine and produce a 3 dimensional map of the path traveled by the capsule.

### Example 49

The capsule of example 1, wherein the digitized output of the 3-axis accelerometer combined with sensed data and time in transit produce a time in gastrointestinal tract segment map of the intestinal tract and provide data supporting the analysis of peristaltic function for each gastrointestinal tract segment.

### Example 50

The capsule of example 1, wherein the electronic components used within the capsule are contained on a series of rigid substrates interconnected with fine pitch flat flexible wiring.

### Example 51

The capsule of example 1, wherein the rigid flex assembly is designed to be folded providing proper alignment of all components and insertion into an outer protective shell.

### Example 52

The capsule of example 1, wherein the folded assembly is inserted into a mold and the outer shell is encapsulated using an FDA approved two-part medium viscosity compound.

### Example 53

The capsule of example 1, wherein a multi-purpose capsule activation stand that communicates with a PC is used prior to capsule use to charge the capsules internal battery, calibrate the capsules accelerometer, calibrate the capsules sensors and test all inter-capsule functions and function as a receiver for the capsule during normal operation.

### Example 54

The capsule of example 53, wherein the multi-purpose capsule activation stand is used to program the capsule. Programmed functions will include setting sensed data thresholds for event triggering such as activating on board cameras, setting the transmit and receive RF channels for the capsules transceiver, setting sensor sample rates and sample rate changes due to sensed data thresholds being met.

### Example 55

The capsule of example 1, wherein external communication with the capsule for both receiving capsule data and controlling the capsules cameras, sensor sampling rate, and power mode are provided using a transceiver having a USB thumb drive form factor connected to a PC.

### Example 56

The capsule of example 55, that uses the external communication with the capsule to define sampling of sensor data in relationship to events detected by the capsule, including but not limited to bleeding, inflammation, abnormalities in pH, abnormalities in motility, and diseases detected including cancers and the like.

### Example 57

The capsule of example 1, wherein external communication with the capsule for both receiving capsule data and controlling the capsule's cameras, sensor sampling rate and power mode are provided using a patient worn small battery powered transceiver and data collection device providing maximum patient mobility. Data collected may be downloaded to a PC upon completion of the test.

### Example 58

The capsule of example 1, wherein said outer shell is formed using an FDA approved silicone rubber encapsulation material or a rigid injection molded polycarbonate 2-piece thin walled casing.

### Example 59

The capsule of example 1, that will provide chemical sensing on a continuous basis in any fluid-containing environment of animals.

### Example 60

The capsule of example 1, wherein said enabling circuitry comprises a switching device.

### Example 61

The capsule of example 3, wherein the said enabling circuitry comprises an electrometric seal about conductive terminals to charge (activate) the battery prior to use.

Although the present invention has been described with respect to one or more particular embodiments, it will be understood that other embodiments of the present invention may be made without departing from the spirit and scope of the present invention. Hence, the present invention is deemed limited only by the appended claims and the reasonable interpretation thereof.

What is claimed is:

1. A device for monitoring an alimentary canal, comprising:
a housing resistant to degradation by alimentary canal fluid;
a first light source for illuminating a first field-of-view of an environment external to the housing;
a first image capture device disposed within the housing, the first image capture device positioned to capture an image of at least a portion of the first field-of-view; and
an analyte sensor configured to obtain at least one measurement of a concentration of analyte in the alimentary canal fluid, the analyte sensor:
a) comprising a sensor substance in a sol-gel material such that the sensor substance reversibly interacts with an analyte of interest, the sensor substance configured to emit electromagnetic energy when the analyte of interest is in contact with the sensor substance and electromagnetic excitation energy is received by the sensor substance, and
b) configured to be in contact with the alimentary canal fluid;
wherein the analyte sensor is configured to generate a trigger signal for controlling the operation of the image capture device.

2. The device of claim 1, wherein the analyte sensor is configured to continuously measure a concentration of analyte in the alimentary fluid.

3. The device of claim 1, wherein the analyte sensor further comprises:
a detector configured to detect electromagnetic energy emitted by the sensor substance; and
a controller in electronic communication with the detector for measuring a concentration of analyte based on the detected electromagnetic energy.

4. The device of claim 3, wherein the first light source provides electromagnetic excitation energy to the sensor substance.

5. The device of claim 3, wherein the analyte sensor further comprises an electromagnetic excitation energy source configured to provide electromagnetic excitation energy to the sensor substance.

6. The device of claim 1, further comprising a transmitter and/or a receiver in electronic communication with the first image capture device.

7. The device of claim 1, further comprising a parametric sensor for measuring a physical parameter of the environment external to the housing, wherein the analyte sensor is configured to generate a trigger signal for controlling the operation of the parametric sensor.

8. The device of claim 7, wherein the physical parameter is sound, pH, temperature, or pressure.

9. The device of claim 1, further comprising:
a second light source for illuminating a second field-of-view of an environment external to the housing; and
a second image capture device disposed within the housing, the second image capture device positioned to capture an image of at least a portion of the second field-of-view.

10. The device of claim 9, wherein the first image capture device and the second image capture device are in electronic communication with the analyte sensor, and one or both of the first image capture device and second image capture device captures an image based on a signal from the analyte sensor.

11. The device of claim 9, further comprising a receiver in electronic communication with the first image capture device and the second image capture device, and wherein one or both of the first image capture device and second image capture device captures an image based on a signal from the receiver.

12. The device of claim 1, further comprising a 3-axis accelerometer.

13. A method of monitoring an alimentary canal, comprising the steps of:
providing a device configured to be disposed in the fluid, the device comprising:
a) a sensor substance in a sol-gel material such that the sensor substance reversibly interacts with an analyte of interest, the sensor substance configured to emit electromagnetic energy when the analyte of interest is in contact with the sensor substance and electromagnetic excitation energy is received by the sensor substance,
b) an electromagnetic energy source capable of emitting light, and
c) a detector configured to detect electromagnetic energy emitted by the sensor substance;
moving the device through at least a portion of the fluid;
exposing the sensor substance to the fluid;
using the detector to make at least one measurement of a property of the electromagnetic energy emitted by the sensor substance;
using a processor to determine at least one analyte concentration value of the fluid based on the at least one measured property; and
generating a trigger signal for controlling the operation of the device.

14. The method of claim 13, wherein the at least one measurement is made continuously.

15. The method of claim 13, further comprising the step of measuring a physical parameter of an environment external to the device by way of a parametric sensor.

16. The method of claim 13, further comprising the step of transmitting the at least one determined analyte concentration value by way of a transmitter.

17. The method of claim 16, further comprising the step of receiving a control signal at the device from a remote transmitter.

18. A method of determining a location of an ingestible device within an alimentary canal, the ingestible device having an analyte sensor, the method comprising the steps of:
using the analyte sensor to determine a concentration of an analyte in a fluid proximate to the device; and
calculating the location within the alimentary canal of the ingestible device based on the determined analyte concentration.

19. The method of claim 18, wherein the ingestible device also has an accelerometer, the method further comprising the steps of:
using the accelerometer to determine a first acceleration of the device;
using the accelerometer to determine a second acceleration of the device, the second acceleration being subsequent to the first acceleration, and the determination of the second acceleration corresponding to the determined analyte concentration;
determining a relative position of the device based on the first and second acceleration; and
wherein the step of calculating the location within the alimentary canal is also based on the determined relative position.

20. A method of locating a region of interest within an alimentary canal using an ingestible device, the ingestible device having an analyte sensor and an accelerometer, the method comprising the steps of:
using the analyte sensor to determine a concentration of an analyte in a fluid proximate to the device;
sending a signal when the analyte sensor senses a concentration of the analyte greater than a pre-determined threshold value, the pre-determined threshold value corresponding to a region of interest; and
using the accelerometer and the analyte sensor to calculate the location of the device within the alimentary canal to determine the location of the region of interest.
